(19) United States
(12) Patent Application Publication
(10) Pub. No.: US 2022/0009857 A1
(43) Pub. Date: Jan. 13, 2022

(54) Multi-Arm Monomolecular White Light-Emitting Materials, Preparation Method and Application Thereof

(71) Applicant: Nanjing University of Posts and Telecommunications, Nanjing (CN)

(72) Inventors: Wenyong LAI, Nanjing (CN); Wan SONG, Nanjing (CN); Xiangchun LI, Nanjing (CN); Yu YAN, Nanjing (CN); Wei HUANG, Nanjing (CN)

(21) Appl. No.: 17/231,489

(22) Filed: Apr. 15, 2021

(30) Foreign Application Priority Data
Jul. 9, 2020 (CN) 202010654240.9

(51) Int. Cl. C07C 15/38; B01J 31/02; C07D 213/06; C07D 251/24; C07D 209/86; C07D 333/08; C07D 265/38; C07D 221/20; H01L 51/00; C07C 255/50; C07C 22/08

(52) U.S. Cl. CPC C07C 15/38; C07C 2603/50; C07D 213/06; C07D 251/24; C07D 209/86; C07D 333/08; C07D 265/38; C07D 221/20; H01L 51/0054; H01L 51/0067; H01L 51/0068; H01L 51/0072; C07C 255/50; C07C 22/08; B01J 31/0239

(57) ABSTRACT

The present invention discloses multi-arm monomolecular white light-emitting materials, preparation method and application thereof. Benzene ring is used as a core, and penta-substituted pyrene and an electron-withdrawing group or an group electron-donating group Ar are used as arms to prepare the multi-arm monomolecular white light-emitting materials; wherein Ar is one of the electron-withdrawing groups such as nitro, cyano, tertiary amine cation, trifluoromethyl, trichloromethyl, sulfonic acid group, formyl, acyl, carboxyl, methoxy, pyridyl, diphenyl sulfone, triazinyl and anthracenedione; or one of the electron-donating groups such as pyrenyl, 9-carbazolyl, 2-thienyl, diphenylamino, tert-butyl diphenylamino, 9-phenoxazinyl, acridinyl, spiro-bifluorenyl, spirofluorenyl acridinyl, alkylamino, dialkylamino, amino and hydroxyl. The present invention simply combines a synthesis method to prepare multi-arm monomolecular white light-emitting materials with novel structure, high fluorescence quantum efficiency, excellent spectrum stability and electroluminescence performance and high color purity, and achieves the preparation of a highly efficient and spectrally stable electroluminescent devices with high color rendering index.

### TECHNICAL FIELD

The present invention belongs to the technical field of optoelectronic materials and applications, and specifically relates to multi-arm monomolecular white light-emitting materials, preparation method and application thereof.

### BACKGROUND

Based on the unique advantages of high brightness, wide viewing angle, low power consumption, wide color gamut, fast response time, good flexibility, wide operating temperature range, simple preparation process and low cost, organic electroluminescent devices have become one of the most promising emerging technologies today and are highly favored by academia and industry. In particular, white organic light emitting diodes are widely used in the field of full-color display and solid-state lighting by virtue of the advantages of light weight, low cost, flexible processing in large area, low energy consumption and excellent warm light source emitting to human body. Generally, a full-spectrum white light emission is mostly achieved by a multicomponent mixed/doped material system or a multi-luminescent layer system. However, the device prepared by the method has proved of poor spectrum stability, interfacial effect between layers, and significantly reduced lifetime, let alone the complicated preparation process. Therefore, how to design and develop a series of highly efficient and spectrally stable organic monomolecular white light-emitting materials has become an urgent problem to be solved.

Most of the currently reported monomolecular white light-emitting materials are polymers, ternary complementary multi-arm molecules of red, green and blue primary colors, binary complementary multi-arm molecules and the like. These white light-emitting polymers and polychromatic multi-arm monomolecular white light-emitting materials are complicated to design and synthesize, and are spectrally unstable and prone to spectral changes at high voltage. Featured by a well-defined chemical structure, easy purification, good solution processing properties, good monodispersity and reproducibility, excellent optoelectronic properties, and clear relationship between structure and properties, non-polychromatic conjugated multi-arm monomolecular white light-emitting materials are a promising organic optoelectronic functional material system of optoelectronic devices. The present invention provides multi-arm monomolecular white light-emitting materials which has the advantages of avoiding phase separation, lower preparation cost of devices, stable electroluminescent spectra and good repeatability. Through simple solution processing, the material can be used as the optoelectronic functional layer material of organic electroluminescent devices, so as to achieve the preparation of a highly efficient and spectrally stable white light-emitting device with high color purity.

### SUMMARY

### Technical Problem

The present invention discloses multi-arm monomolecular white light-emitting materials, preparation method and application thereof as an organic luminescent layer material of white organic electroluminescent devices. The material is a promising organic optoelectronic functional material because of its function of solving the problems of insufficient variety, poor spectral stability, low luminescence efficiency and low color purity of monomolecular white light-emitting material, effectively avoiding phase separation, simplifying the preparation process of devices, reducing their preparation cost, and achieving the preparation of a highly efficient and spectrally stable white light-emitting device with high color rendering index.

### Technical Solution

The present invention discloses multi-arm monomolecular white light-emitting materials, preparation method and application thereof as an organic luminescent layer material of white light-emitting organic electroluminescent devices. The materials are prepared from the raw material of pentabromobenzene containing Ar functional group and 1-pyrenyl boronic acid ester through Suzuki coupling reaction. The advantages of the present invention lie in that the organic monomolecular white light-emitting material avoids the problems of phase separation and spectrum stability of a multi-luminescent layer system or a multicomponent mixed/doped material system in achieving a full-spectrum emission, and the problems of unstable spectra of monomolecular polymers or multi-color molecules, which are difficult to purify, reduces the preparation cost of devices, and has good reproducibility. In addition, the material has already been used to prepare a highly efficient and spectrally stable white light electroluminescent device with high color purity.

The present invention provides a series of multi-arm monomolecular white light-emitting materials characterized by having the following general structural formula:

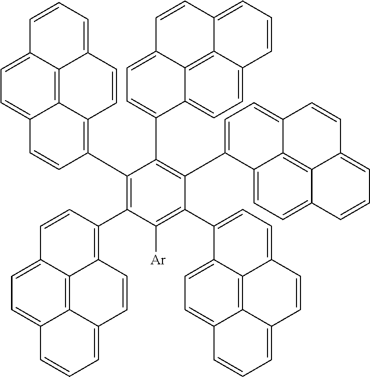

in the general structural formula, multi-arm monomolecular white light-emitting materials are prepared from a benzene ring as a core, penta-substituted pyrene and Ar as arms, and functional groups, wherein Ar is one of the electron-withdrawing groups such as bromine, fluorine, nitro, cyano, tertiary amine cation, trifluoromethyl, trichloromethyl, sulfonic acid group, formyl, acyl, carboxyl, methoxy, pyridyl, diphenyl sulfone, triazinyl and anthracenedione, and has the following specific structural formulas:

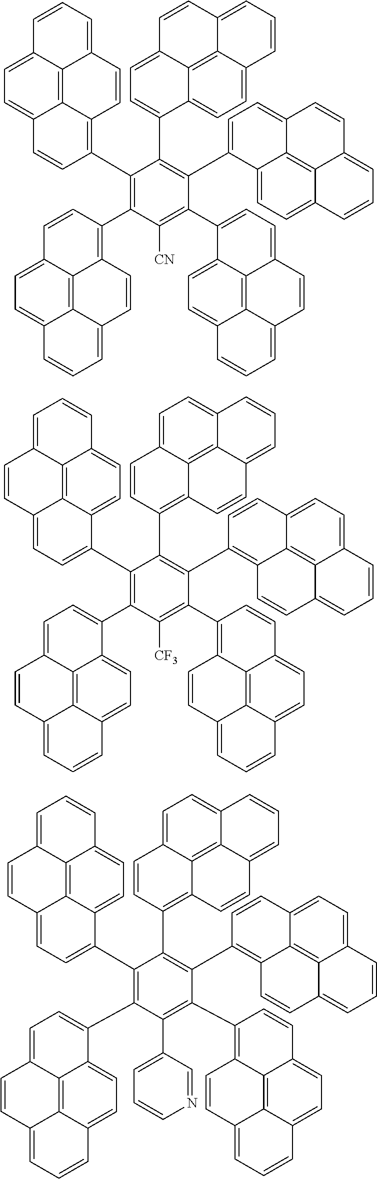

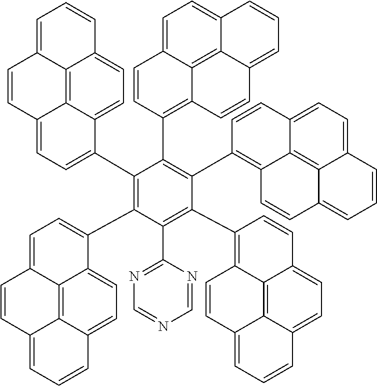 [SMILES: c1cc2ccc3ccc(-c4c(-c5ncncn5)c(-c5ccc6ccc7cccc8ccc5c6c78)c(-c5ccc6ccc7cccc8ccc5c6c78)c(-c5ccc6ccc7cccc8ccc5c6c78)c4-c4ccc5ccc6cccc7ccc4c5c67)c4ccc(c1)c2c34]

when Ar is one of the electron-donating groups such as pyrenyl, 9-carbazolyl, 2-thienyl, diphenylamino, tert-butyl diphenylamino, 9-phenoxazinyl, acridinyl, spiro-bifluorenyl, spirofluorenyl acridinyl, alkylamino, dialkylamino, amino and hydroxyl, it has the following specific structural formulas:

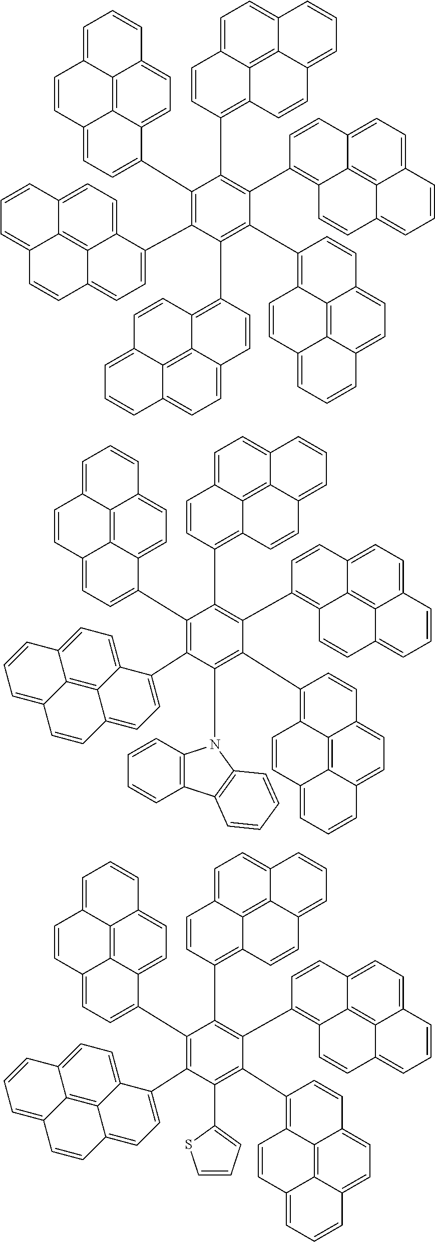

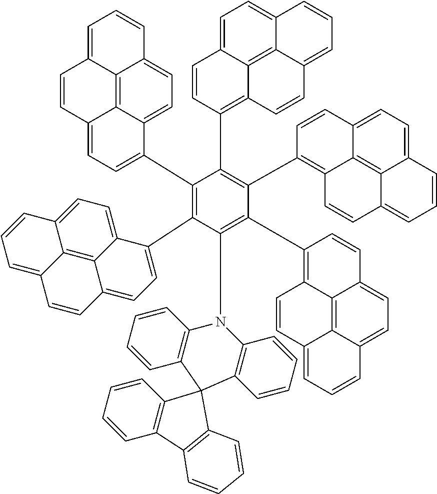 [SMILES: c1ccc2c(c1)-c1ccccc1C21c2ccccc2N(c2c(-c3ccc4ccc5cccc6ccc3c4c56)c(-c3ccc4ccc5cccc6ccc3c4c56)c(-c3ccc4ccc5cccc6ccc3c4c56)c(-c3ccc4ccc5cccc6ccc3c4c56)c2-c2ccc3ccc4cccc5ccc2c3c45)c2ccccc21]

The preparation method of the multi-arm monomolecular white light-emitting materials according to the present invention is characterized in that a series of multi-arm monomolecular white light-emitting materials are prepared from the raw material of pentabromobenzene containing Ar functional group (Ph5Br—Ar) and 1-pyrenyl boronic acid ester (molar ratio: 1:9-1:15) through Suzuki coupling reaction in a dark place under nitrogen atmosphere.

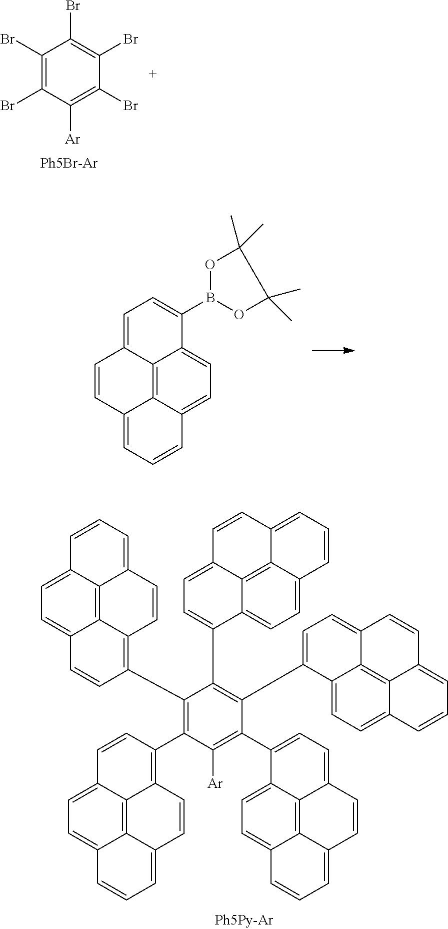

Specifically, the preparation method of a multi-arm monomolecular white light-emitting material comprises the following steps:

step 1: mixing a reactant of pentabromobenzene containing Ar functional group (Ph5Br—Ar) and 1-pyrenyl boronic acid ester, a catalyst tetrakis (triphenylphosphine) palladium, and a phase transfer catalyst tetrabutylammonium bromide in a dark place under nitrogen atmosphere, dissolving in potassium carbonate and toluene, wherein the volume ratio of potassium carbonate to toluene is 1:(2-3), and the molar mass ratio of tetrakis (triphenylphosphine) palladium catalyst to phase transfer catalyst tetrabutylammonium bromide to Ph5Br—Ar is (0.2-0.4):(0.1-0.3):1, and reacting at 90-110° C. for 24-72 h in a dark place; and step 2: after the reaction, cooling to room temperature, extracting the resulting mixed solution with an organic solvent and a saturated salt solution, drying the separated organic layer, performing suction filtration, separating and purifying the mixture obtained by concentrating the solution through column chromatography, and drying to obtain a target product.

The present invention also provides a use of the multi-arm monomolecular white light-emitting materials as an organic luminescent layer material in optoelectronic fields including white light organic electroluminescent devices.

### Advantageous Effect

The present invention provides a series of multi-arm monomolecular white light-emitting materials, which can be used as an organic luminescent layer material of white organic light emitting diode devices. According to the present invention, the efficient preparation of material is achieved through ingenious design and coupling reaction. The materials are a promising organic optoelectronic functional material system because of novel and well-defined structure, excellent spectral stability and thermal stability, high fluorescence quantum efficiency, and adjustable optical and electrochemical properties achieved by selecting different functional groups. Besides, the material system effectively solves the problems of insufficient variety, poor spectral stability, low luminescence efficiency and low color purity of monomolecular white light-emitting material, avoids phase separation, simplifies the preparation process of devices, reduces their preparation cost, and achieves the preparation of a highly efficient and spectrally stable white light-emitting device with high color rendering index.

### BRIEF DESCRIPTION OF THE FIGURES

FIG. 1 shows the cyclic voltage-current curves (CV curves) of compounds A, B, and C.

FIG. 2 shows the thermal weight loss curves of compounds A, B, E, F, and H.

FIG. 3 shows the luminance-voltage curves of compounds B, D and G.

FIG. 4 shows the powder X-ray diffraction patterns of compounds A, B, C, D, E, F, G, and H.

FIG. 5 is the fluorescence emission spectra of compound B.

FIG. 6 is the mass spectra of compound A.

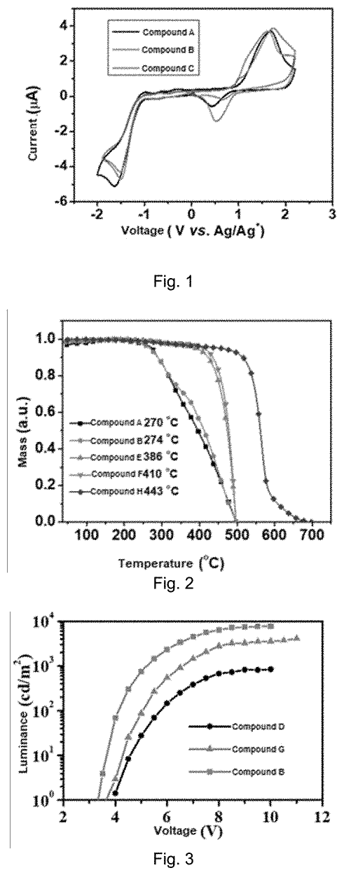

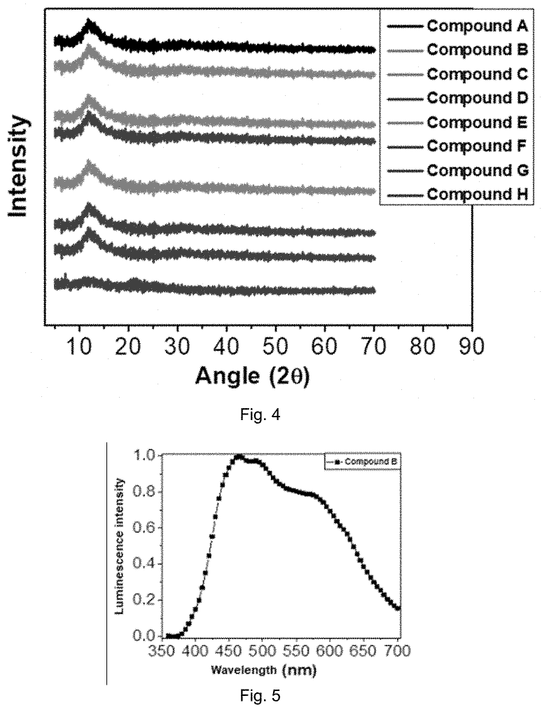

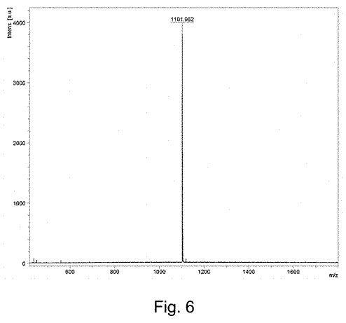

### DESCRIPTION OF THE PRESENT INVENTION

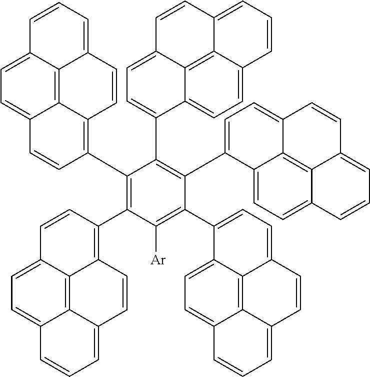

A multi-arm monomolecular white light-emitting material has the following general structural formula:

In the general structural formula, a multi-arm monomolecular white light-emitting material is prepared from a benzene ring as a core, penta-substituted pyrene and Ar as arms, and functional groups, wherein Ar is one of the electron-withdrawing groups such as bromine, fluorine, nitro, cyano, tertiary amine cation, trifluoromethyl, trichloromethyl, sulfonic acid group, formyl, acyl, carboxyl, methoxy, pyridyl, diphenyl sulfone, triazinyl and anthracenedione; or one of the electron-donating groups such as pyrenyl, 9-carbazolyl, 2-thienyl, diphenylamino, tert-butyl diphenylamino, 9-phenoxazinyl, acridinyl, spiro-bifluorenyl, spirofluorenyl acridinyl, alkylamino, dialkylamino, amino and hydroxyl.

Based on the preparation method of the multi-arm monomolecular white light-emitting material, a series of multi-arm monomolecular white light-emitting materials are prepared from the raw material of pentabromobenzene containing Ar functional group (Ph5Br—Ar) and 1-pyrenyl boronic acid ester (molar ratio: 1:9-1:15) through Suzuki coupling reaction in a dark place under nitrogen atmosphere.

### Example 1

Preparation of Compound A

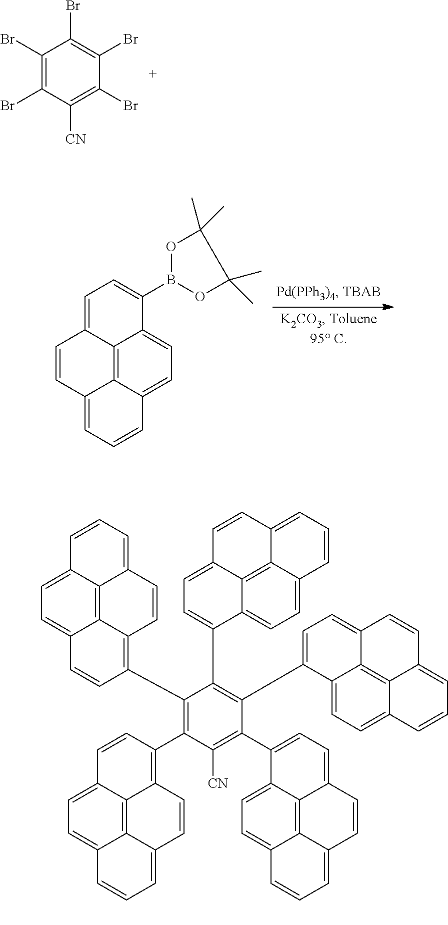

Ph5Br—CN (500 mg, 1.02 mmol), 1-pyrenyl boronic acid ester (3.33 g, 10.15 mmol) and tetrabutylammonium bromide (TBAB) (98.17 mg, 0.30 mmol) were added to a 100 mL two-necked flask, sealed, wrapped in foil paper to avoid light, and pumped with nitrogen for three times. Then, a catalyst tetrakis (triphenylphosphine) palladium Pd (PPh₃)₄ (351.9 mg, 0.30 mmol) was added rapidly to the flask that was pumped with nitrogen for three times. Finally, the bubbled toluene (24 mL) and 2M K₂CO₃ solution (8 mL) were injected into the reaction flask and reacted at 95° C. for 48 h. After the reaction, the solution was extracted with the organic solvent DCM and saturated salt solution for three times to separate an organic layer, which was dried with MgSO₄. The mixture obtained by concentrating the solution after suction filtration was separated and purified by column chromatography, and dried in a vacuum drying oven to obtain the product Ph5Py-CN (714 mg, yield: 63.8%). MALDI-TOF-MS (m/z): calcd for C₈₇H₄₅N, Molecular Weight: 1104.32, Exact Mass: 1103.36, Found: 1101.96 (M⁺).

### Example 2

Preparation of Compound B

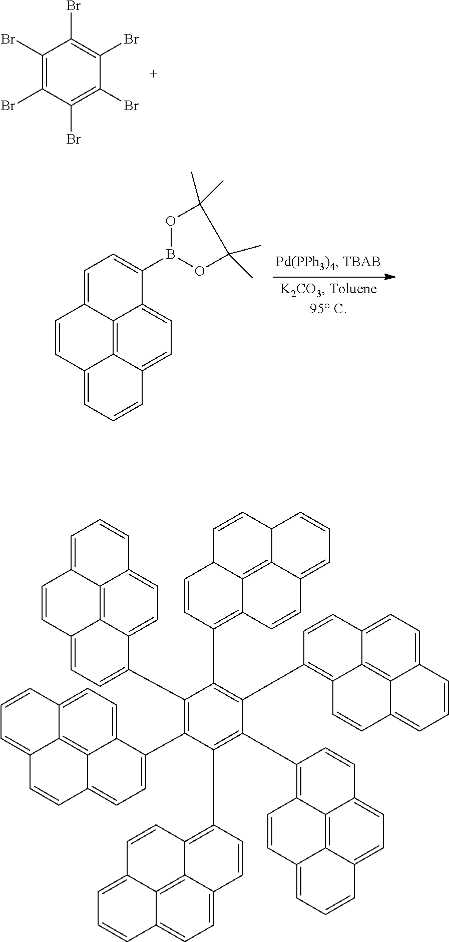

Hexabromobenzene (500 mg, 0.91 mmol), 1-pyrenyl boronic acid ester (3.57 g, 10.88 mmol) and tetrabutylammonium bromide (TBAB) (87.7 mg, 0.27 mmol) were added to a 100 mL two-necked flask, sealed with rubber stoppers, wrapped in foil paper to avoid light, and pumped with nitrogen for three times. Then, a catalyst Pd (PPh₃)₄ (314.3 mg, 0.27 mmol) was added rapidly to the flask that was pumped with nitrogen for three times. Finally, the bubbled toluene (24 mL) and 2M K₂CO₃ aqueous solution (8 mL) were injected into the reaction flask and reacted at 95° C. for 48 h. After the reaction, the solution was extracted with the organic solvent DCM and saturated salt solution for three times to separate an organic layer, which was dried with MgSO₄. The crude product obtained by concentrating the solution after suction filtration was separated and purified by column chromatography, and dried in a vacuum drying oven to obtain the product Ph6Py (670 mg, yield: 72.8%). MALDI-TOF-MS (m/z): calcd for C₁₀₂H₅₄, Molecular Weight: 1279.55, Exact Mass: 1278.42, Found: 1276.65 (M⁺).

### Example 3

Preparation of Compound C

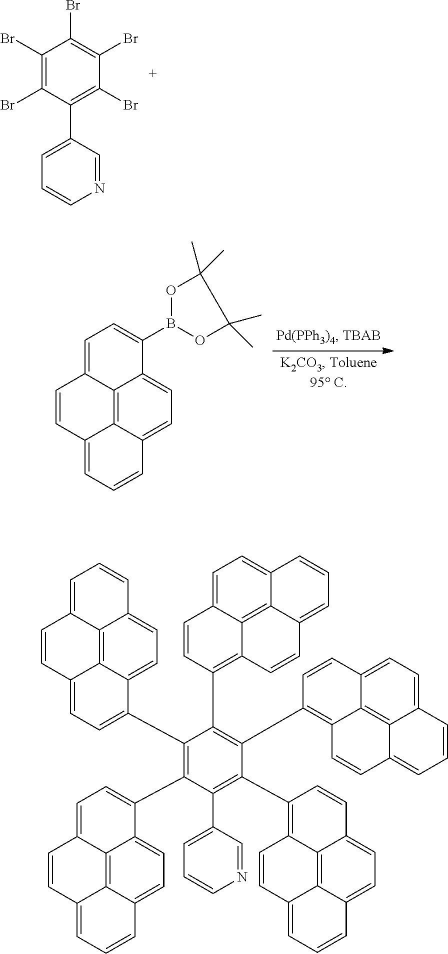

Ph5Br-Py (500 mg, 0.92 mmol), 1-pyrenyl boronic acid ester (3.57 g, 9.19 mmol) and tetrabutylammonium bromide (TBAB) (74.0 mg, 0.23 mmol) were added to a 100 mL two-necked flask, sealed with rubber stoppers, wrapped in foil paper to avoid light, and pumped with nitrogen for three times. Then, a catalyst Pd (PPh₃)₄ (265.4 mg, 0.23 mmol) was added rapidly to the flask that was pumped with nitrogen for three times. Finally, the bubbled toluene (24 mL) and 2M K₂CO₃ aqueous solution (8 mL) were injected into the reaction flask and reacted at 95° C. for 48 h. After the reaction, the solution was extracted with the organic solvent DCM and saturated salt solution for three times to separate an organic layer, which was dried with MgSO₄. The crude product obtained by concentrating the solution after suction filtration was separated and purified by column chromatography, and dried in a vacuum drying oven to obtain the product Ph6Py (780 mg, yield: 73.6%). MALDI-TOF-MS (m/z): calcd for C₉₁H₄₉N, Molecular Weight: 1156.40, Exact Mass: 1155.39, Found: 1155.92 (M⁺).

### Example 4

Preparation of Compound D

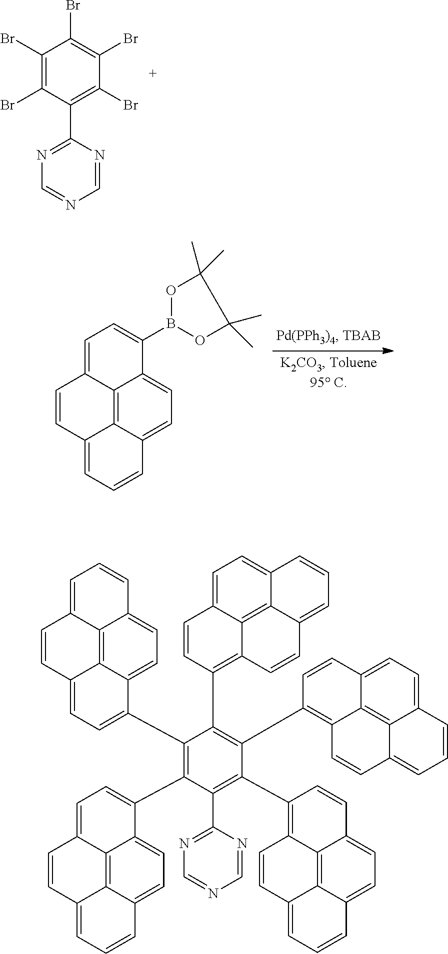

Ph5Br-Tz (500 mg, 0.91 mmol), 1-pyrenyl boronic acid ester (3.00 g, 9.15 mmol) and tetrabutylammonium bromide (TBAB) (73.7 mg, 0.23 mmol) were added to a 100 mL two-necked flask, sealed with rubber stoppers, wrapped in foil paper to avoid light, and pumped with nitrogen for three times. Then, a catalyst Pd(PPh₃)₄ (264.3 mg, 0.23 mmol) was added rapidly to the flask that was pumped with nitrogen for three times. Finally, the bubbled toluene (24 mL) and 2M K₂CO₃ aqueous solution (8 mL) were injected into the reaction flask and reacted at 95° C. for 48 h. After the reaction, the solution was extracted with the organic solvent DCM and saturated salt solution for three times to separate an organic layer, which was dried with MgSO₄. The crude product obtained by concentrating the solution after suction filtration was separated and purified by column chromatography, and dried in a vacuum drying oven to obtain the product Ph6Py (692 mg, yield: 65.3%). MALDI-TOF-MS (m/z): calcd for C₈₉H₄₇N₃, Molecular Weight: 1158.38, Exact Mass: 1157.38, Found: 1158.22 (M⁺).

### Example 5

Preparation of Compound E

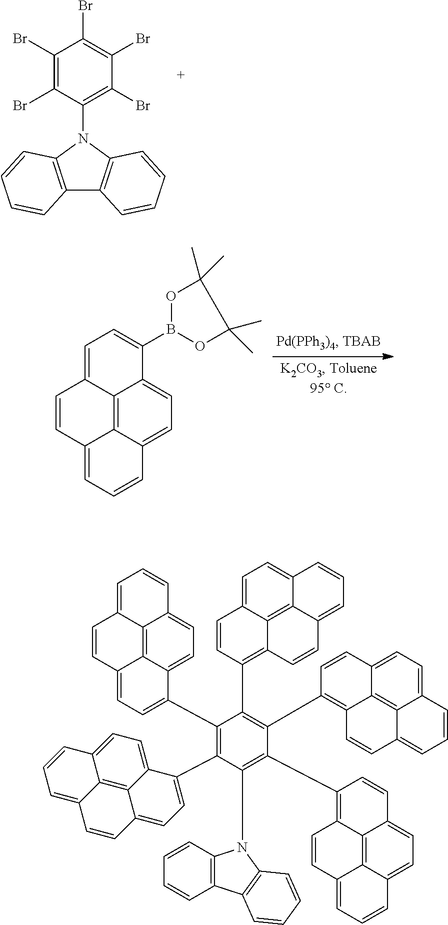 [SMILES: Brc1c(Br)c(Br)c(-n2c3ccccc3c3ccccc32)c(Br)c1Br ; "+" ; CC1(C)OB(c2ccc3ccc4cccc5ccc2c3c45)OC1(C)C ; c1cc2ccc3ccc(-c4c(-c5ccc6ccc7cccc8ccc5c6c78)c(-c5ccc6ccc7cccc8ccc5c6c78)c(-n5c6ccccc6c6ccccc65)c(-c5ccc6ccc7cccc8ccc5c6c78)c4-c4ccc5ccc6cccc7ccc4c5c67)c4ccc(c1)c2c34]

Ph5Br-Cz (500 mg, 0.79 mmol), 1-pyrenyl boronic acid ester (2.59 g, 7.90 mmol) and tetrabutylammonium bromide (TBAB) (63.7 mg, 0.23 mmol) were added to a 100 mL two-necked flask, sealed with rubber stoppers, wrapped in foil paper to avoid light, and pumped with nitrogen for three times. Then, a catalyst Pd (PPh₃)₄ (228.3 mg, 0.20 mmol) was added rapidly to the flask that was pumped with nitrogen for three times. Finally, the bubbled toluene (24 mL) and 2M K₂CO₃ aqueous solution (8 mL) were injected into the reaction flask and reacted at 95° C. for 48 h. After the reaction, the solution was extracted with the organic solvent DCM and saturated salt solution for three times to separate an organic layer, which was dried with MgSO₄. The crude product obtained by concentrating the solution after suction filtration was separated and purified by column chromatography, and dried in a vacuum drying oven to obtain the product Ph6Py (687 mg, yield: 70.0%). MALDI-TOF-MS (m/z): calcd for C₉₈H₅₃N, Molecular Weight: 1244.51, Exact Mass: 1243.42, Found: 1143.96 (M⁺).

### Example 6

Preparation of Compound F

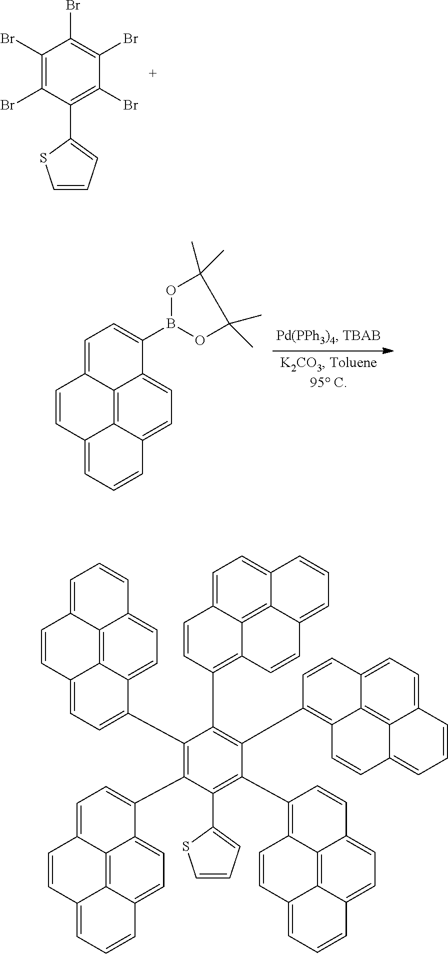

Ph5Br-Tp (500 mg, 0.91 mmol), 1-pyrenyl boronic acid ester (2.99 g, 9.10 mmol) and tetrabutylammonium bromide (TBAB) (73.3 mg, 0.23 mmol) were added to a 100 mL two-necked flask, sealed with rubber stoppers, wrapped in foil paper to avoid light, and pumped with nitrogen for three times. Then, a catalyst Pd (PPh₃)₄ (262.8 mg, 0.23 mmol) was added rapidly to the flask that was pumped with nitrogen for three times. Finally, the bubbled toluene (24 mL) and 2M K₂CO₃ aqueous solution (8 mL) were injected into the reaction flask and reacted at 95° C. for 48 h. After the reaction, the solution was extracted with the organic solvent DCM and saturated salt solution for three times to separate an organic layer, which was dried with MgSO₄. The crude product obtained by concentrating the solution after suction filtration was separated and purified by column chromatography, and dried in a vacuum drying oven to obtain the product Ph6Py (850 mg, yield: 80.2%). MALDI-TOF-MS (m/z): calcd for C₉₀H₄₈S, Molecular Weight: 1161.43, Exact Mass: 1160.35, Found: 1161.26 (M⁺).

### Example 7

Preparation of Compound G

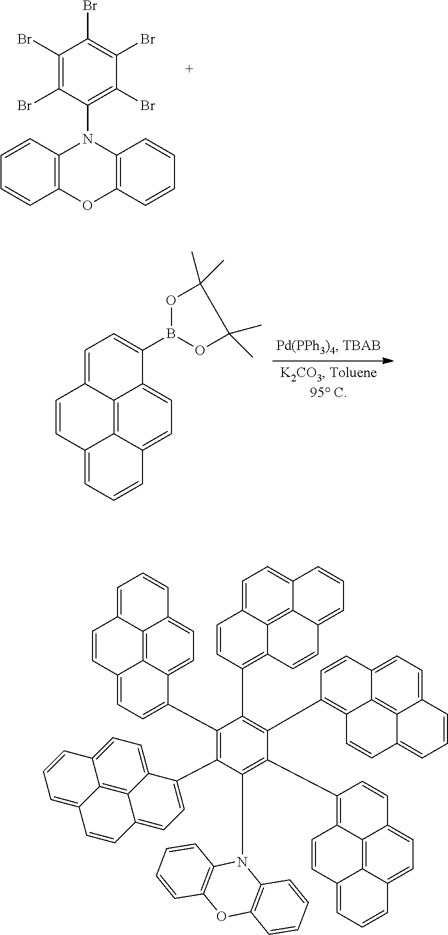 [SMILES: Brc1c(Br)c(Br)c(N2c3ccccc3Oc3ccccc32)c(Br)c1Br ; "+" ; CC1(C)OB(c2ccc3ccc4cccc5ccc2c3c45)OC1(C)C ; c1ccc2c(c1)Oc1ccccc1N2c1c(-c2ccc3ccc4cccc5ccc2c3c45)c(-c2ccc3ccc4cccc5ccc2c3c45)c(-c2ccc3ccc4cccc5ccc2c3c45)c(-c2ccc3ccc4cccc5ccc2c3c45)c1-c1ccc2ccc3cccc4ccc1c2c34]

Ph5Br—Pa (500 mg, 0.77 mmol), 1-pyrenyl boronic acid ester (2.53 g, 7.71 mmol) and tetrabutylammonium bromide (TBAB) (62.1 mg, 0.19 mmol) were added to a 100 mL two-necked flask, sealed with rubber stoppers, wrapped in foil paper to avoid light, and pumped with nitrogen for three times. Then, a catalyst Pd (PPh₃)₄ (222.7 mg, 0.19 mmol) was added rapidly to the flask that was pumped with nitrogen for three times. Finally, the bubbled toluene (24 mL) and 2M K₂CO₃ aqueous solution (8 mL) were injected into the reaction flask and reacted at 95° C. for 48 h. After the reaction, the solution was extracted with the organic solvent DCM and saturated salt solution for three times to separate an organic layer, which was dried with MgSO₄. The crude product obtained by concentrating the solution after suction filtration was separated and purified by column chromatography, and dried in a vacuum drying oven to obtain the product Ph6Py (682 mg, yield: 70.3%). MALDI-TOF-MS (m/z): calcd for C₉₈H₅₃NO, Molecular Weight: 1260.51, Exact Mass: 1259.41, Found: 1260.16 (M⁺).

### Example 8

Preparation of Compound H

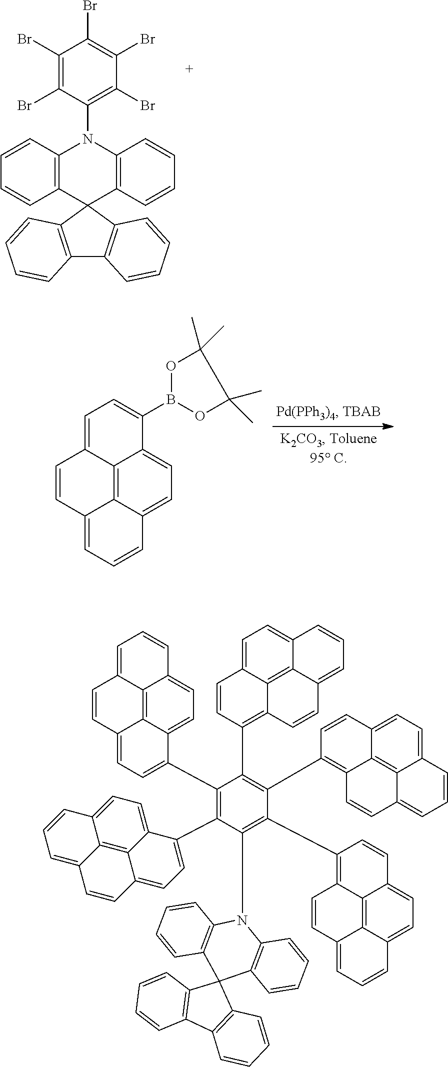

Ph5Br-spiro AC (500 mg, 0.63 mmol), 1-pyrenyl boronic acid ester (2.06 g, 6.28 mmol) and tetrabutylammonium bromide (TBAB) (50.6 mg, 0.16 mmol) were added to a 100 mL two-necked flask, sealed with rubber stoppers, wrapped in foil paper to avoid light, and pumped with nitrogen for three times. Then, a catalyst Pd (PPh₃)₄ (181.3 mg, 0.16 mmol) was added rapidly to the flask that was pumped with nitrogen for three times. Finally, the bubbled toluene (24 mL) and 2M K₂CO₃ aqueous solution (8 mL) were injected into the reaction flask and reacted at 95° C. for 48 h. After the reaction, the solution was extracted with the organic solvent DCM and saturated salt solution for three times to separate an organic layer, which was dried with MgSO₄. The crude product obtained by concentrating the solution after suction filtration was separated and purified by column chromatography, and dried in a vacuum drying oven to obtain the product Ph6Py (643 mg, yield: 72.8%). MALDI-TOF-MS (m/z): calcd for C₁₁₁H₆₁N, Molecular Weight: 1408.72, Exact Mass: 1407.48, Found: 1407.86 (M⁺).

### Example 9: Preparation of OLED Device

ITO glass was ultrasonically cleaned and treated with oxygen plasma to obtain the square resistance of 10 Ω/cm². The hole injection layer was PEDOT or PVK, and the luminescent layer was any one of compound A, compound B, compound C, compound D, compound E, compound F, compound G, or compound H, which were amorphous and had good film-forming property. Both the hole injection layer and the luminescent layer were coated by spinning. The cathode electrode was Ca/AI or UFA, respectively; wherein the minimum start-up voltage of the OLED device prepared based on compound B was 3.12 V and the maximum luminance was 8865 cd/m².

In addition to several cases described in the above embodiments, the following conditions shall fall within the applicable scope of the present invention by reason of similar preparation method as the above embodiments; that is, Ar is one of the electron-withdrawing groups such as bromine, fluorine, nitro, cyano, tertiary amine cation, trifluoromethyl, trichloromethyl, sulfonic acid group, formyl, acyl, carboxyl, methoxy, pyridyl, diphenyl sulfone, triazinyl and anthracenedione; or one of the electron-donating groups such as pyrenyl, 9-carbazolyl, 2-thienyl, diphenylamino, tert-butyl diphenylamino, 9-phenoxazinyl, acridinyl, spiro-bifluorenyl, spirofluorenyl acridinyl, alkylamino, dialkylamino, amino and hydroxyl.

The description of the embodiments of the inventor is only for a better understanding of the present invention. It should be noted that the present invention is not limited to these embodiments, and any equivalent transformations made according to the technical solutions of the present invention are within the protection scope of the present invention.

1. A multi-arm monomolecular white light-emitting material, characterized by having the following general structural formula: 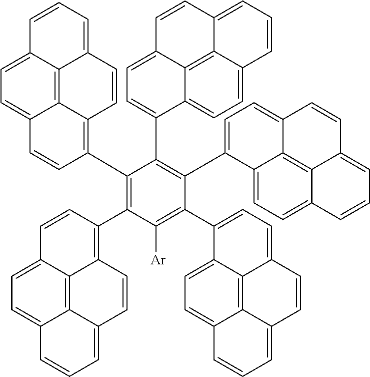
In the general structural formula, a benzene ring is used as a core, and penta-substituted pyrene and Ar are used as arms, wherein Ar is an electron-withdrawing group or an electron-donating group.

2. The multi-arm monomolecular white light-emitting material according to claim 1, characterized in that Ar is selected from one of the electron-withdrawing groups such as bromine, fluorine, nitro, cyano, tertiary amine cation, trifluoromethyl, trichloromethyl, sulfonic acid group, formyl, acyl, carboxyl, methoxy, pyridyl, diphenyl sulfone, triazinyl and anthracenedione.

3. The multi-arm monomolecular white light-emitting material according to claim 1, characterized by having the following structural formulas when Ar is an electron-withdrawing group including cyano, trifluoromethyl, pyridyl and triazinyl:

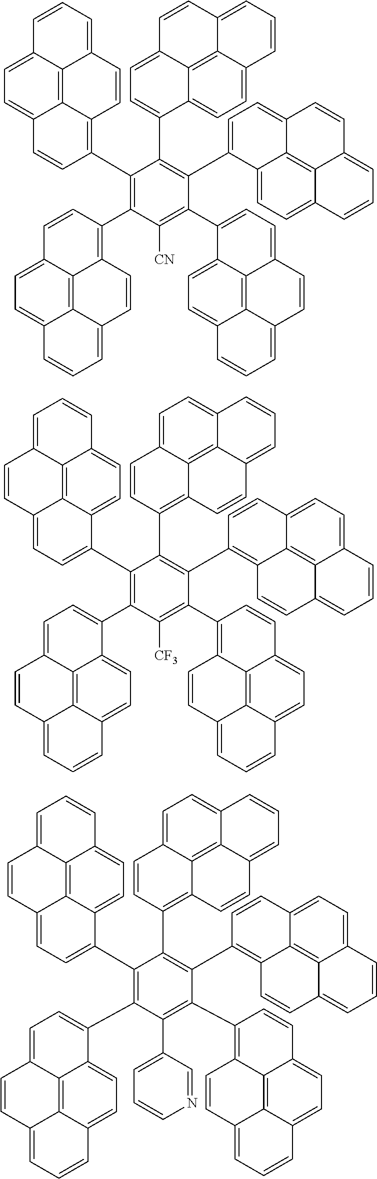

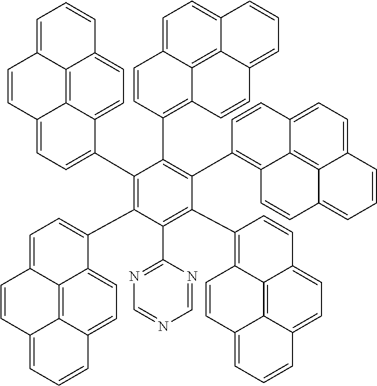 [SMILES: c1cc2ccc3ccc(-c4c(-c5ncncn5)c(-c5ccc6ccc7cccc8ccc5c6c78)c(-c5ccc6ccc7cccc8ccc5c6c78)c(-c5ccc6ccc7cccc8ccc5c6c78)c4-c4ccc5ccc6cccc7ccc4c5c67)c4ccc(c1)c2c34]

4. The multi-arm monomolecular white light-emitting material according to claim 1, characterized in that Ar is selected from one of the electron-donating groups such as pyrenyl, 9-carbazolyl, 2-thienyl, diphenylamino, tert-butyl diphenylamino, 9-phenoxazinyl, acridinyl, spiro-bifluorenyl, spirofluorenyl acridinyl, alkylamino, dialkylamino, amino and hydroxyl.

5. The multi-arm monomolecular white light-emitting material according to claim 1, characterized by having the following structural formulas when Ar is an electron-donating group including pyrenyl, 9-carbazolyl, 2-thienyl and spirofluorenyl acridinyl:

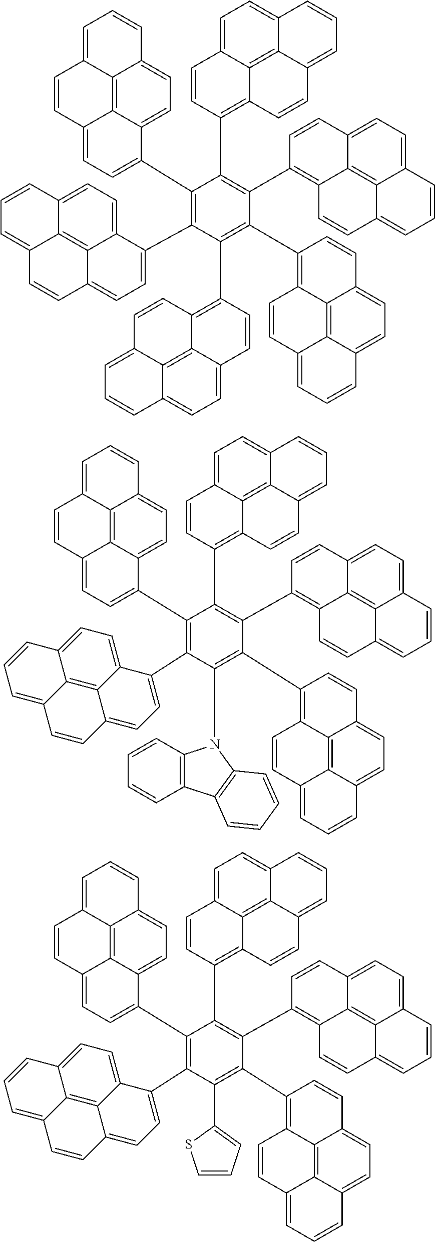

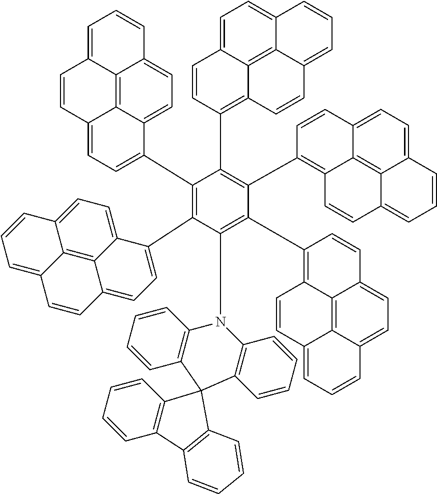 [SMILES: c1ccc2c(c1)-c1ccccc1C21c2ccccc2N(c2c(-c3ccc4ccc5cccc6ccc3c4c56)c(-c3ccc4ccc5cccc6ccc3c4c56)c(-c3ccc4ccc5cccc6ccc3c4c56)c(-c3ccc4ccc5cccc6ccc3c4c56)c2-c2ccc3ccc4cccc5ccc2c3c45)c2ccccc21]

6. A preparation method of a multi-arm monomolecular white light-emitting material according to any of claim 1, characterized in that the multi-arm monomolecular white light-emitting material is prepared from the raw material of pentabromobenzene containing Ar functional group (Ph5Br—Ar) and 1-pyrenyl boronic acid ester through Suzuki coupling reaction, with the reaction equation as follows:

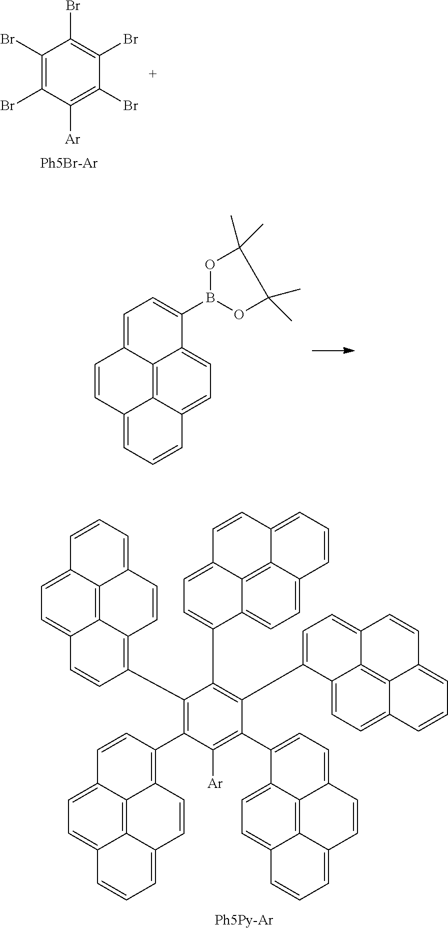

7. The preparation method of a multi-arm monomolecular white light-emitting material according to claim 6, characterized by comprising the following steps:
step 1: mixing a reactant of Ph5Br—Ar and 1-pyrenyl boronic acid ester, a catalyst tetrakis (triphenylphosphine) palladium, and a phase transfer catalyst tetrabutylammonium bromide in a dark place under nitrogen atmosphere, dissolving in potassium carbonate and toluene, and reacting at 90-110° C. for 24-72 h in a dark place; and
step 2: after the reaction, cooling to room temperature, extracting the resulting mixed solution with an organic solvent DCM and a saturated salt solution, drying the separated organic layer, performing suction filtration, separating and purifying the mixture obtained by concentrating the solution through column chromatography, and drying to obtain a target product.

8. The preparation method of a multi-arm monomolecular white light-emitting material according to claim 6, characterized in that the molar ratio of Ph5Br—Ar to 1-pyrenyl boronic acid ester is 1:9-1:15.

9. The volume ratio of potassium carbonate to toluene is 1:(2-3), and the molar mass ratio of tetrakis (triphenylphosphine) palladium catalyst to phase transfer catalyst tetrabutylammonium bromide to Ph5Br—Ar is (0.2-0.4):(0.1-0.3):1.

10. A use of a multi-arm monomolecular white light-emitting material according to claim 1, characterized in that the material is used as a functional layer material of optoelectronic devices including organic electroluminescent devices, flexible electronic devices and stretchable electronic devices.
